(19) United States
(12) Patent Application Publication
(10) Pub. No.: US 2010/0105999 A1
(43) Pub. Date: Apr. 29, 2010

(54) Graphical User Interface for Glucose Monitoring System

(75) Inventors: Lynn Dixon, Fremont, CA (US); Jonathan Fern, Alameda, CA (US); Juli Goldstein, Oakland, CA (US); Nicole J. Walker, Okland, CA (US); Samuel Mason Curry, San Francisco, CA (US); William Matievich, JR., Alameda, CA (US); John Holland, Sausalito, CA (US); Daniel Khalili, San Ramon, CA (US)

Correspondence Address: Abbott Diabetes Care Inc.;Bozicevic, Field & Francis LLP, 1900 University Ave, Suite 200, East Palo Alto, CA, 94303, US

(73) Assignee: Abbott Diabetes Care Inc.

(21) Appl. No.: 12/622,310

(22) Filed: Nov. 19, 2009

Related U.S. Application Data
(63) Continuation of application No. 12/233,584, filed on Sep. 18, 2008.

(51) Int. Cl. A61B 5/145

(52) U.S. Cl. 600/347

(57) ABSTRACT

Graphical user interfaces (GUIs) for a medical device such as a blood glucose meter, systems and methods are provided. Embodiments include GUIs in which a user may navigate between icons and select certain display outputs which summarize all or certain glucose data over particular time periods. In certain embodiments, animated icons and/or graphs and/or text summaries may be provided.

### CROSS REFERENCE TO RELATED APPLICATIONS

This application is related to U.S. patent application Ser. No. 10/837,886 published as US2005/0245844; and Ser. No. 10/899,773 published as US2006/0024774; and Ser. Nos. 11/830,760; 11/830,779; 11/830,786; 11/831,706; 11/535,985; 11/535,986 published as US2007/0079783; Ser. No. 11/035,131 published as 2005/0164322, Ser. No. 11/831,649 published as 2008/0033318, Ser. Nos. 11/868,762, 12/035,348, 11/830,770, 10/701,993 published as 2004/0138588, Ser. Nos. 12/185,116; 12/185,117; and 12/185,118; as well as PCT applications PCT/US2005/014855; WO2006/19665; WO/2008/39946; and WO/2008/39949; and EP1779109; which are each assigned to the same assignee as the present application and are hereby incorporated by reference.

### BACKGROUND OF THE INVENTION

Self Care and Self Monitoring of Blood Glucose (SMBG) are central to the management of diabetes. Based on recommendations of a treating health care professional, a person with diabetes will be instructed to use SMBG to check glucose levels and make appropriate adjustments to medication regimens, activity, diet or other behaviors. FIG. 1 illustrates the tools typically used in conventional SMBG, including from left to right a lancing device 2 including a removable and replaceable cap 4, a glucose meter 6 and a container 8 of glucose testing strips. The self care regimen includes testing the glucose level periodically, sometimes multiple times each day. The conventional process typically involves:

1—locating a new sterile lancet;
2—loading a lancet into the lancing device 2;
3—locating & opening test strip vial 8;
4—removing a test strip and replacing the lid on the vial 8;
5—inserting the test strip into the meter 6;
6—coding/calibrating the meter 6;
7—priming the lancing device 2;
8—lancing the finger or alternate site;
9—applying blood to the test strip;
10—waiting for the reading;
11—disposing of the used test strip;
12—replaceing the cap 4 on the lancet 2;
13—disposing of the used lancet; and
14—disposing of the test strip.

SMBG systems are provided by Abbott Diabetes Care Inc., which is the assignee of the present application, including the Freestyle® and Precision® analyte monitoring lines of products. As monitoring analytes such as glucose continues to be of interest to those managing their health, it is desired to have a reliable and efficient analyte (e.g., glucose) testing system that is easy to use and which requires minimal steps each time a person tests his or her analyte level (e.g., a person with diabetes tests his or her glucose level), while not sacrificing accuracy, reliability nor functionality.

### SUMMARY OF THE INVENTION

A graphical user interface (GUI) is provided for a portable medical device, such as an analyte monitoring device (e.g., a glucose meter), as well as devices, systems, methods and kits. In further describing the invention, embodiments will be described primarily as glucose—related devices, systems and methods, where such description is for exemplary purposes only and is in no way intended to limit the invention. It will be appreciated that embodiments of the invention may be employed in a variety of medical devices, for example a variety of analyte monitoring devices.

Embodiments include meters that include an electronic display and one or more user input buttons, as well as processor-readable code embedded in a digital memory for programming a processor to generate and display a temporary user-readable output based on stored glucose data. The temporary display may include multiple icons configured for manipulation by a user. The GUI may be configured to be user-navigable between the icons and user-selectable among multiple configurations of the icons to produce a selected display output which summarizes all or certain glucose data over a particular time period.

In one aspect, the selected display output includes a scrolling word summary on a portion of a display of the portable glucose meter that is smaller than the complete scrolling word summary. The scrolling word summary may include a query and/or a statement of fact relating to status of health of a user based on one or more relationships between the glucose data and one or more target glucose levels. The summary statements support self-management through providing pattern recognition directly to users.

In another aspect, the glucose data may be configurable by the user through the manipulation of icons to selectably add metadata relating to conditions of particular glucose measurements including one or more temporal relationships to one or more meals, insulin boluses, or exercise events, or time period of a day, or combinations thereof, such that the selected display output is configurable by the user to summarize only certain glucose data corresponding to selected conditions.

In another aspect, a target range of glucose levels for the user may be configurable by the user. The selected display output may include a graph showing multiple glucose measurements plotted over the time period delineating those measurements inside and outside of the target range.

In a graph according to certain embodiments, a bubble icon may be generated and displayed which contains a summary of certain glucose data when those certain glucose data are highlighted or otherwise selected within the graph on the display. The summary contained in the bubble may include one or more glucose levels along with metadata relating to conditions of one or more glucose measurements corresponding to certain highlighted or otherwise selected glucose data. The metadata may include one or more temporal relationships to one or more meals, insulin boluses, or exercise events, or time period of a day, or combinations thereof.

In a graph according to certain embodiments, the delineating may include applying different shading and/or color to a region of the graph representing glucose levels within the target range compared with one or more regions of the graph representing glucose levels outside the target range. The graph may map to a calendar image with delineated glucose ranges to help users understand fluctuations in their blood glucose readings over time.

One or more instructive still and/or video icons may be generated and displayed to indicate current status of a glucose measurement and/or a next step for a user to take in a glucose measurement. An icon may be generated and displayed that illustrates a testing site of the body of a user in proximity with the portable glucose meter indicating to the user that a next step is to begin a glucose test by lancing the skin of the user. One or more icons may be generated and displayed to indicate that the meter has detected initial body fluid flow into a testing strip and/or that a testing strip contains adequate body fluid to conduct an assay.

One or more power-up sequences of the portable medical device may be provided. The sequences may include ones with and without preparation for and expectation of performing a glucose measurement.

Logbook entries of glucose measurements may be generated and displayed that are selectable for reviewing specific details relating to the glucose measurements.

A series of reminder icons and/or screens may be generated and displayed to output one or more alarms to a user to remind the user to measure glucose or check or inject insulin at a certain time or within a certain amount of time, or combinations thereof. Automatic reminders to test again after a blood glucose measurement may indicate a glucose level that is below a selected target range or when the measurement is indicated as before meals helps users to comport with evidence based clinical practice guidelines that recommend re-testing a certain amount of time, e.g., 15 minutes, after a hypoglycemic event or a certain amount of time, e.g., 2 hours, after eating (i.e., post-prandial).

A method of operating a processor-based portable diabetes care apparatus that has a GUI as set forth above and below herein are also provided. In addition, processor-based portable diabetes care devices that include the GUI are also provided herein.

### BRIEF DESCRIPTION OF THE DRAWINGS

FIG. 1 illustrates a conventional glucose monitoring system including separate lancing device, lancets, glucose meter and testing strips.

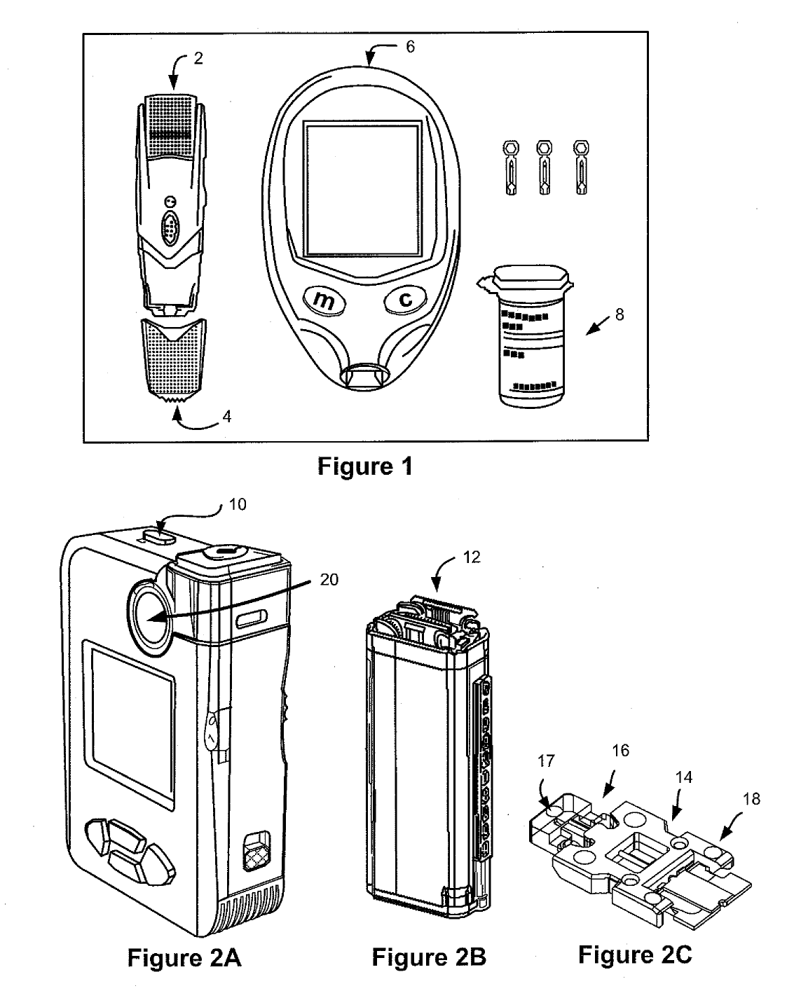

FIGS. 2a-2c illustrate an advantageous glucose monitoring system including integrated glucose meter with cartridge containing combination strips with lancets according to certain embodiments.

FIG. 3 illustrates a menu tree for overall operation of a graphical user interface of a glucose monitoring system according to certain embodiments.

FIGS. 4a-4q illustrate display screens of a graphical user interface for two power up options for testing and analysis of a glucose monitoring system according to certain embodiments.

FIGS. 5a-5i illustrate display screens of a graphical user interface for three test flow sequences of a glucose monitoring system according to certain embodiments.

FIGS. 6a-6p illustrate display screens of a graphical user interface for four further test flow sequences with markings options of a glucose monitoring system according to certain embodiments.

FIGS. 7a-7f illustrate display screens of a graphical user interface for two further test flow sequences wherein Lo and Hi blood sugar levels have been determined by a glucose monitoring system according to certain embodiments.

FIGS. 8a-8f illustrate display screens of a graphical user interface for a SetUp Wizard for a glucose monitoring system according to certain embodiments.

FIGS. 9a-9k illustrate display screens of a graphical user interface for analysis of glucose monitoring results of a glucose monitoring system according to certain embodiments.

FIGS. 10a-10h, as well as FIG. 6n, illustrate display screens of a graphical user interface for providing reminders for a glucose monitoring system according to certain embodiments.

FIGS. 11a-11j illustrate display screens of a graphical user interface for adjusting settings of a glucose monitoring system according to certain embodiments, including setting date and time, target glucose ranges, and/or meal markers, reviewing summaries of glucose monitoring results and/or changing language of preference.

FIGS. 12a-12n illustrate display screens of a graphical user interface for using certain tools such as for running control tests, running SetUp Wizard and running diagnostics for a glucose monitoring system according to certain embodiments.

### DETAILED DESCRIPTION OF THE EMBODIMENTS

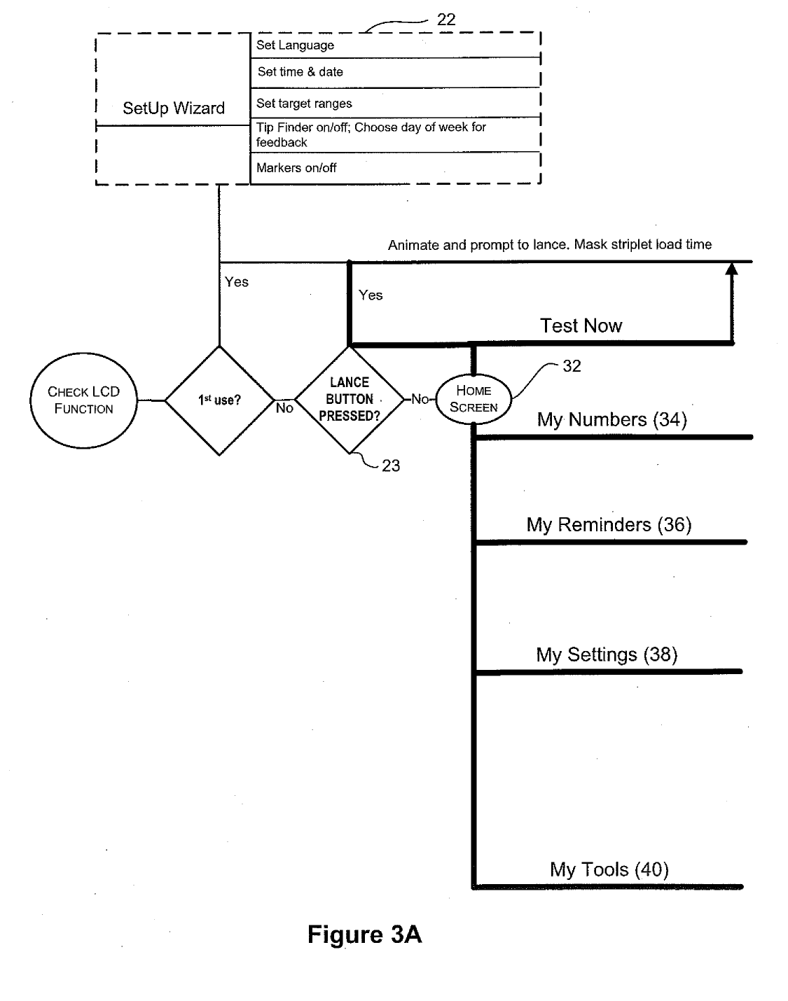

Integrated lancing and testing systems have been described by Abbott Diabetes Care, the same assignee as the present application, which are described at U.S. application Ser. Nos. 11/535,985, 11/870,420 and 11/535,986 and other US and international patent applications including U.S. Ser. Nos. 11/830,779, 11/831,649, 11/160,427, 10/899,773, 10/701,993, 11/146,897, 11/160,407, 11/350,398, 10/629,348, 11/830,770, 11/868,762, 12/035,348, 11/035,131, 10/837,886, 11/830,760, 11/830,786, and 11/831,706, the disclosures of which are herein incorporated by reference. FIGS. 2A, 2B and 2C illustrate an embodiment of an integrated self-care analyte system FIG. 2A illustrates an example of an integrated analyte meter 10 having a cartridge such as that illustrated at FIG. 2B loaded therein. The meter 10 includes a display and one or more user input features such as buttons that may include one or more press buttons, wheel buttons, track ball buttons, touch screen buttons, dials and/or keys, and/or a voice actuation microphone. The cartridge 12 may contain many strips such that the SMBG regimen involves loading the cartridge far less than each time a person with diabetes tests his or her glucose level. FIG. 2C illustrates a strip 14 including both a lancet 16 with a cap 17 and a glucose testing component 18.

Once the cartridge is loaded, the process of testing a glucose level with the system illustrated at FIGS. 2A, 2B and 2C is simplified to:

1—pressing a test button 20 (see FIG. 2A);
2—lancing a finger or alternate site (blood may be applied to the strip automatically after lancing, i.e., without user performing additional action);
3—waiting for the test result; and
4—disposing of the test strip 14 (which is automatically re-capped within the meter 10)

It is desired to both simplify the SMBG process further and provide additional functionality with a uniquely advantageous graphical user interface for an integrated diabetes system that fully supports self-management of diabetes.

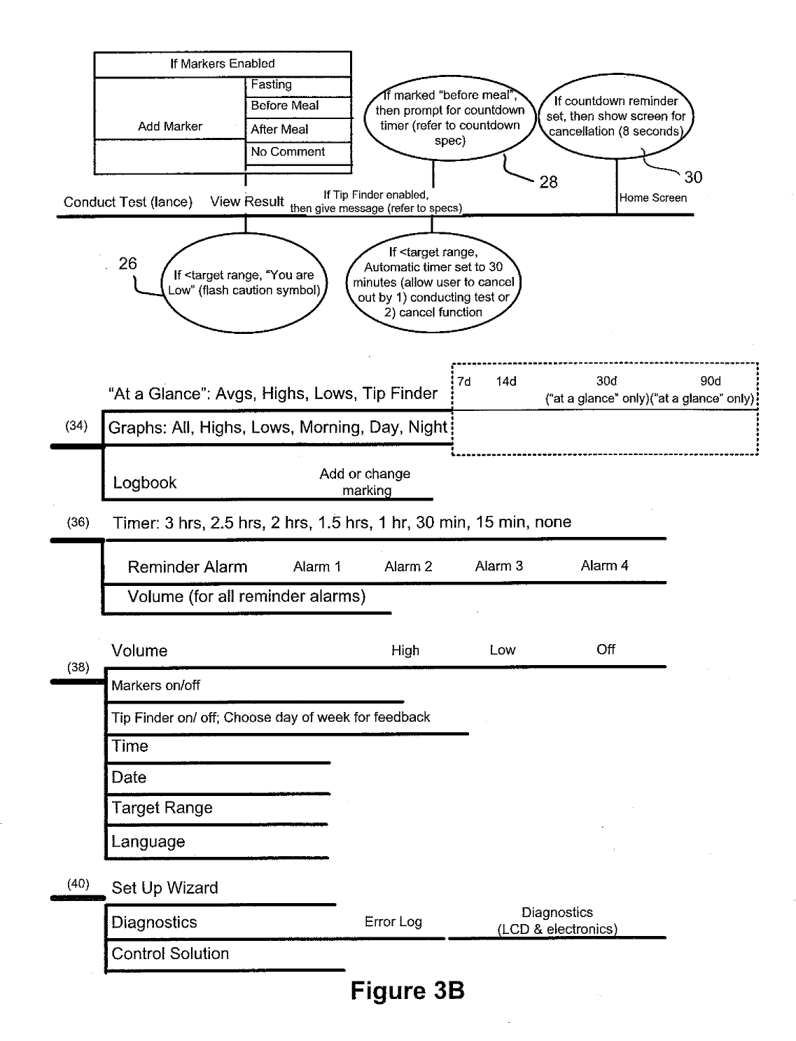

An exemplary menu tree for a graphical user interface or GUI for an advantageous glucose self care testing apparatus is illustrated at FIG. 3. The menu tree illustrated at FIG. 3 includes a SetUp Wizard™ 22 where a user can set various parameters, e.g., the language, date, and time, and target glucose level ranges, and the like.

The user can also use the SetUp Wizard to turn on or off a summary finder or periodic feedback relating to glucose test results. The summary finder can include summary information of a test or tests that appears on the screen or is otherwise output to the user at a predetermined time and/or day. Messages may include emoticons and other icons and/or messages, e.g.: for in range numbers: a happy face icon and a message “your fasting numbers last week were in your range”, or the like. For high numbers, a warning or danger signal icon with the message “all of your before meal numbers have been above your range last week”, or the like, may appear. For low numbers, a warning or danger signal icon may appear with message “all of your morning numbers have been low this week”, or the like. For the situation where some numbers are in range and others not, a happy face or danger icon may appear depending on the ratio with a message “x out of y test have been in your range this week and the others were all high”, or the like. For the situation where some numbers are low and others in range, a happy face or danger icon may appear depending on the ratio with the message “your were low 5 out of the 20 times you last tested”, or the like. The SetUp Wizard can be used to customize the summary finder, e.g., to include all numbers, or just fasting numbers, or before or after meal numbers, or morning, day or night numbers, and the time period can be set, e.g., 1 day, 3 day, 7 day, 14 day, 30 days, 90 days, etc.

Once set up, the system is ready to perform a test. A user can press a lance button 23. The SetUp Wizard may also permit the user to turn markers on and off. Markers may be enabled 24 to indicate certain conditions under which glucose tests were performed such as proximity before, during or after a meal, meal contents or recent eating habits, exercise information, time of day or night, insulin dosages and time/date or dosage, among others. Results of the test may be viewed 26 including the actual glucose level along with a message such as you are low or high or in range along with an appropriate icon. If “before meal” is marked, then a countdown timer may be prompted 28, or a countdown reminder may be set which may show a screen for cancellation 30.

A user may navigate to a home screen 32 in the GUI which is certain embodiments may provide options to go to “My Numbers” 34, “My Reminders” 36, “My Settings” 38 and/or “My Tools” 40 in navigating the GUI. In My Numbers 34, a user may review an “At a Glance” screen that may show averages, highs, lows, summary finders or a manual or automatic summary information ticker or scrollable message. Also in My numbers 34, a user may view graphs, such as those that will be discussed in more detail with reference to FIGS. 16a-16b. The My Numbers section may also include a logbook, such as an electronic version of paper logbooks such as those that persons living with diabetes have been keeping since before any electronic logbooks were available. Markings may be added or changed in the logbook by the user.

In My Reminders 36, a user may set a timer at 5, 10, 15, 30, or 45 minutes, or 1 hour, 2 hours, 3 hours, or days or weeks. In use, the timer may be used for events such as testing, and also meals and exercise and insulin dosing may be set by a user with the timer. Alarms with different sounds may be set to different timers by the user, and volumes may be set.

In My Settings 38, a user may set volume, markers on/off, summary finder or summary information on/off, including choosing day and/or time for the GUI to provide feedback. Time, date, target ranges and languages may be set in My Settings. In My Tools 40, a user can get back to the original SetUp Wizard 22, and can perform diagnostics and control solution tests.

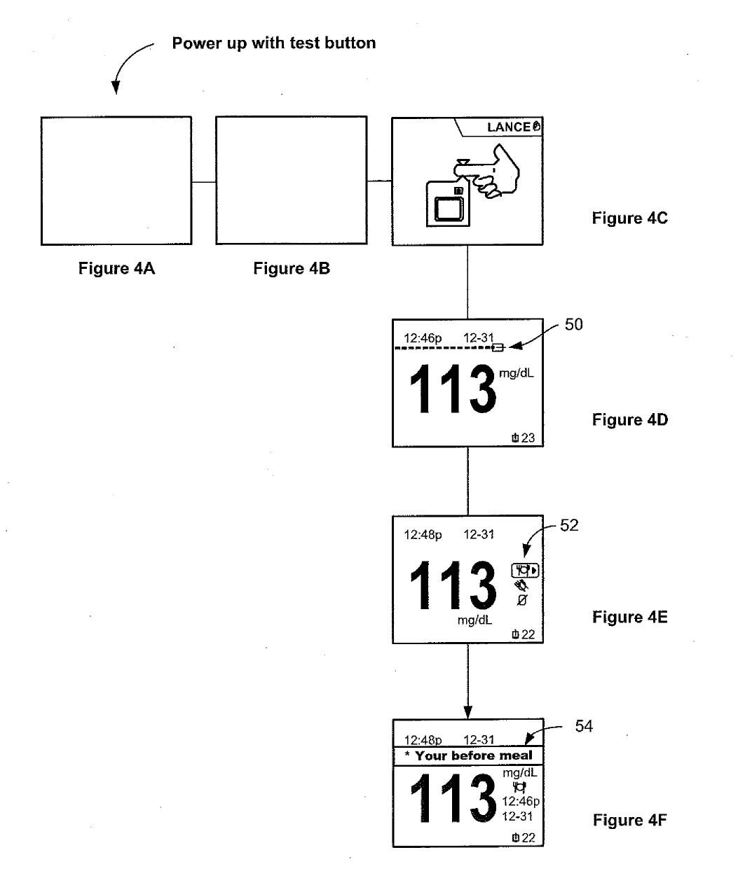

Navigation within a GUI in accordance with certain embodiments is illustrated and discussed with reference to FIGS. 4a-4q. In such embodiments, the system may include various options of use, depending on the task required. For example, in certain embodiments, the system may have various optional configurations depending on whether a test is to be performed, or only access to data is desired, etc. FIGS. 4a-4f illustrate a power up of the integrated meter with a test button. The user may power up according to this option when intending to test his or her glucose level. FIGS. 4g-4q illustrate a power up of the integrated meter with power button. The user may power up according to his second option when not intending to test his or her glucose level. The functionalities of navigating the GUI, accessing information stored on the meter and setting up the GUI using My Numbers, My Reminders, My Setting and My Tools are accessible with either power up option.

In the power up with test button, FIG. 4a shows a starting screen which moves to FIG. 4b indicating the system is preparing to navigate the user through a test by loading a test device such as a test strip and/or lancet and/or a combines lancet and test device such as a Striplet™ testing device (a Striplet device is a device that integrates a lancet and a test device as a unit (see for example U.S. patent application Ser. No. 11/535,986, published as US2007/0149897, the disclosure of which is herein incorporated by reference). In further describing the embodiments below, embodiments using an integrated lancet/test device such as a Striplet™ device will be used for exemplary purposes only and is in no way intended to limit the scope of the invention. It will be appreciated that medical devices that use separate lancets and testing devices (such as test strips) may be used, as well as those that use integrated lancets and test devices like Striplets™ or otherwise. A Striplet test device may be extracted from an installed cartridge within a meter for lancing to be followed by testing. FIG. 4c illustrates a screen that appears when the meter is ready, indicating to the user that he or she may initiate lancing as soon as the user places the skin location to be lanced at the skin receiving surface of the meter. In addition to the image on the screen, an optional light around the test button may flash to indicate to the user that the meter is ready to lance. In the example of FIG. 4c, the meter is arranged for lancing and testing at a finger site, while alternative site testing may also be used, i.e., at a location of a person's body different than a finger. When the user is ready, he or she may press the lance button which initiates lancing and testing as well as recapping the lancet and ejecting the used Striplet automatically. Also not illustrated at FIG. 4c, a blood droplet may appear on the screen that begins to fill when the meter senses at a sample application indicator, e.g., an electrode or the like, that body fluid has begun to fill the test strip, and may be indicated to be full when the meter senses at a fill indicator, e.g., a fill electrode, or the like, that the testing chamber is filled. In certain embodiments, adequate sample volume may be a small volume, e.g., less than about 1 microliter, e.g., less than about 0.5 microliters, less than about 0.3 microliters, less than about 0.1 microliters in certain embodiments.

The glucose test result will appear on a next screen as illustrated at FIG. 4d. FIG. 4d includes video icon 50 indicating to the user that a Striplet is in the process of being recapped and ejected. In one embodiment, the Striplet actually ejects from the meter when the Striplet reaches the edge of the screen, and the testing result number appears on the screen at about the same time. In other words, a moving graphics or video shows the testing process by moving across the display. The ejection (and also re-capping if present) are illustrated when the representation of the testing unit is moved to an edge of the display. Thereafter, FIG. 4e shows that the number of S triplets remaining has been decremented by one from “23” in FIG. 4d to “22” in FIG. 4e. FIG. 4e also illustrates that meal markers 52 have been set. FIG. 4f illustrates a summary ticker 54 that provides some information beyond just the glucose level indicated as “113” that the test indicated, e.g., the summary ticker may provide information determined from comparing the test result number to target ranges and/or meal markers. At this point in the power up with test button process, the user may go to screens illustrated at any of FIGS. 4h-4q.

Screens 4h-4q are also accessible in a power up with power button process that does not involve testing and that does not even require that a test is now possible, e.g., there might not be any strips or lancets or Striplets (i.e., combined strip/lancets) in the meter, or the loaded strips may be damaged, or some other restriction may cause the meter not to permit a test as determined using meter diagnostics. In the absence of those circumstances restricting testing, a user may execute or press a “test now” icon or button and start a test even though the system was powered up using power button. There may also be conditions like expired strip or invalid date and time that may involve user confirmation to proceed with a test.

FIG. 4i indicates that a user may select My Numbers and get a screen illustrated at FIG. 4j allowing the user to select At A Glance, Graphs and Logbook. FIG. 4k indicates that a user may select My Reminders and get a screen illustrated at FIG. 41 allowing the user to select reminders and alarms. FIG. 4m indicates that a user may select My Settings and get a screen illustrated at FIG. 4n allowing the user to set volume, time, date, markers, summary finder, summary, target range and/or language, wherein a user may have to scroll to access one or more of the selectable items as illustrated at FIG. 4o. FIG. 4p indicates that a user may select My Tools and get a screen illus traced FIG. 4q allowing the user to manage a control test of the meter and/or Striplets using a control solution, or to access the SetUp Wizard or diagnostics.

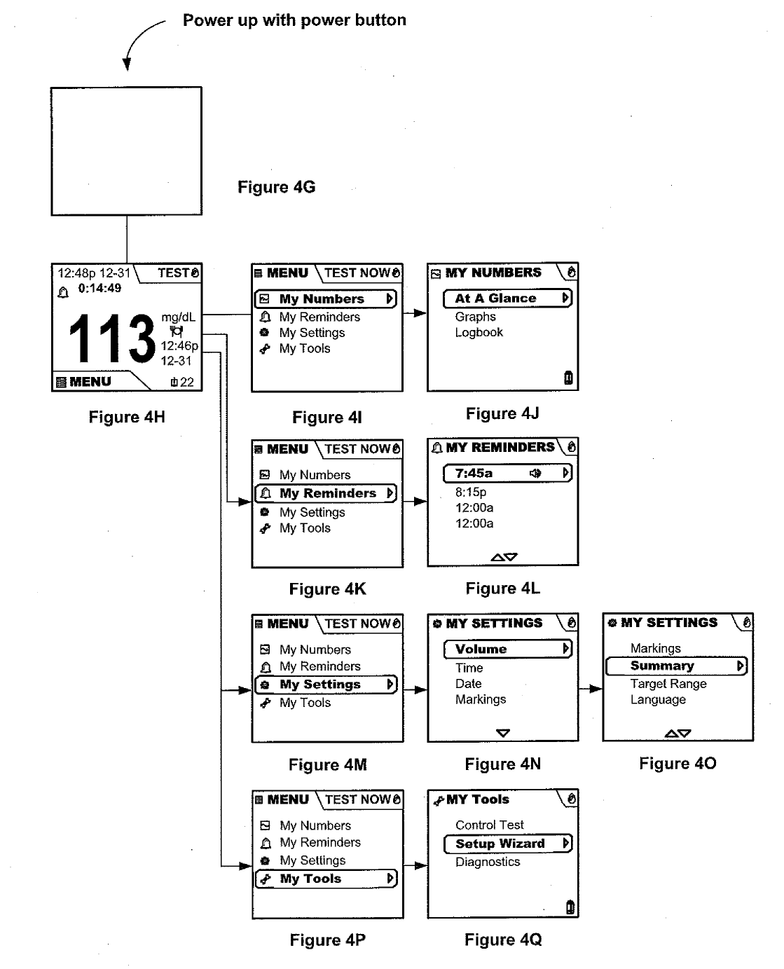

FIGS. 5a-5i, 6a-6p and 7a-7f illustrate functions of a GUI in accordance with certain embodiments. FIG. 5a illustrate a screen showing date 56, time 58, a recently-measured glucose level of “113 mg/dL” 60, and the number of Striplets left in the cartridge 62. A user will want to check the date and time shown on this screen to be sure it matches the real date and time, otherwise the records of the meter will be off, as will markers, alarms and summaries of information that may be depend on the time of the day or day of the week. The meter may be configured to be able to detect if the meter has last time/date. If the meter recognizes that the time/date is invalid, the meter may be configured to notify the user. FIG. 5a indicates that a just-used Striplet is about to be ejected from the meter after being recapped inside the meter. FIG. 5b illustrates a screen just after ejection of the used Striplet. The date and time 64 of the test resulting in the glucose level shown in the provided screen of FIG. 5b, as well as the current time 56 and date 58. At this point, the user may access the menu by clicking at 66 to get to My Numbers, My reminders, My Settings and My Tools. The user may also initiate another test by clicking at 68.

FIG. 5c shows the screen of FIG. 5a, except that this time summary finder is on. Once the just-used S triplet is ejected from meter, then a screen is illustrated at FIG. 5d showing a ticker or scrolling message or video text appears. The scrolling messages may relate to glucose level history and provide queries. The GUI can be arranged to provide the scrolling messages after each test, after a specified number of tests, after certain types of tests (e.g., mornings, after meals), periodically, or upon request of the user.

Examples of messages that may scroll across the screen include, but are not limited to, the following:

“My Range: 80 to 180. My mornings were in range last week.”
“Better this week than last.”
“80 to 180. Your numbers have improved this week.”
“Below 80. A lot of lows last week in the mornings.”
“Felt unwell? Lots of lows last week.”
“Something different? Low morning results last week.”
“You were running high in the mornings last week.”
“Levels in the mornings have been above my range.”

Examples of messages that may appear in 14 day summaries include the following:

“My mornings have been in range last 14 days.”
“80 to 180. You were in range before meals in the last 14 days.”
“Feeling ok? A lot of morning lows in last two weeks.”
“Make any changes? You are running high before meals.”
“You have had a lot of results above your range in the last two weeks.”
“Last two weeks—Your levels have been high during the day.”

Examples of message that may appear in 30 day summaries include the following:

“Your fastings were in range last month. Did you notice a difference?”
“Taken care of your lows? You improved last month.”
“You had several highs in the last 30 days, especially in the mornings.”
“Feeling unwell? You had low readings at night last month.”
“Your range: 80 to 180. In the last 30 days, you have been above your range after meals.”

The next screen illustrated at FIG. 5e is comparable to that of FIG. 5b.

In another sequence, FIG. 5f is the same screen as FIGS. 5a and 5c, except this time before and after meal markers 70 and 72, respectively, at FIG. 5g have been set. FIG. 5h indicates that a before meal marker has been set to indicate that the blood glucose result was conducted before eating. A reminder 76 to test again, for example 15 or 30 minutes, or one, two or three hours, after eating (configurable by the user) will appear visibly on the screen and with an audible alarm at the user configured hours following the meal. Once the reminder time has been set for post-meal testing, the user-configured time will appear as the default for future blood glucose tests indicated as before eating. FIG. 5i illustrates that a 15 minute clock 78 is counting down after a hypoglycemic event (i.e., below a user-configured target range). The user is expected to retest 80 before the clock 78 gets to zero.

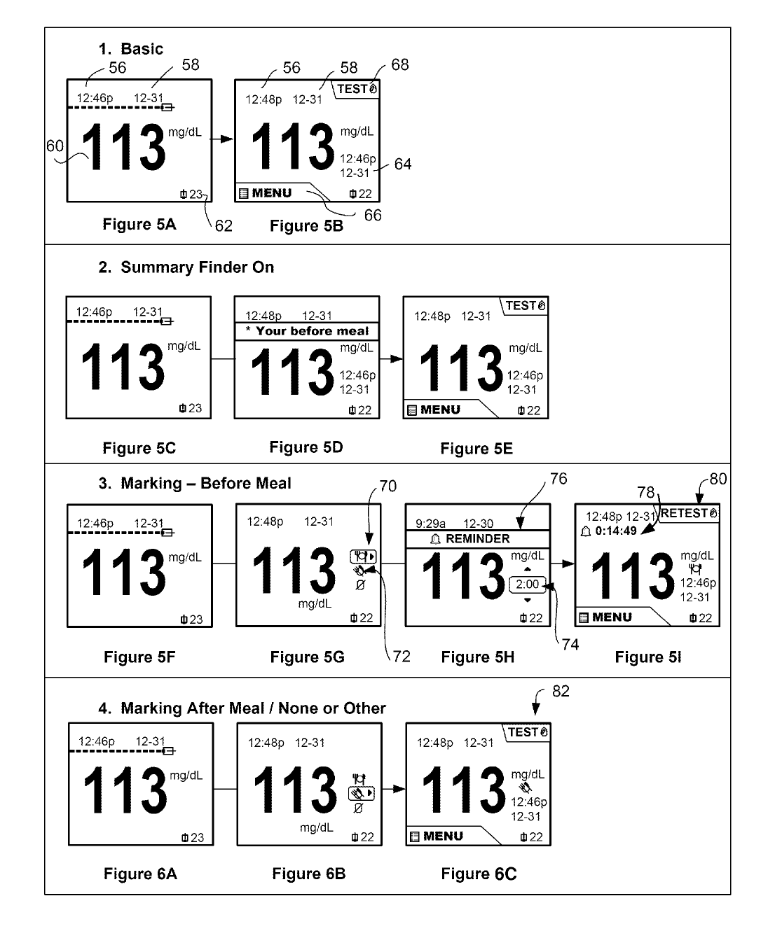

FIGS. 6a, 6b and 6c illustrate a sequence of after meal marking or none. FIG. 6c indicates that the meter is ready to test 82.

FIGS. 6d-6g illustrate a sequence of screens in a GUI in accordance with certain embodiments wherein a low glucose level is measured. The initial screen shows the low result 84 which is “75 mg/dL” while the used, recapped S triplet is about to be ejected from the meter. FIG. 6e illustrates a next screen in which a caution symbol 86 is shown and may even be flashing. A message 88 indicates that the user has low blood sugar, e.g., saying “YOU ARE LOW”. A 15 minute countdown is shown beginning at FIG. 6f during which time the user is expected to take action to raise his or her blood sugar, or perhaps simply wait to test again when the blood sugar may be measured to be higher. The user is expected to retest 90 as illustrated at FIG. 6g. The feature has been designed to support automation of evidence based clinical practice guidelines to re-test after a hypoglycemic event.

FIGS. 6h-6k illustrate a sequence of screens in a GUI in accordance with certain embodiments with an after meal marking and summary finder each on. FIG. 6i indicates that selection of an after meal marker 92. FIG. 6j illustrates a summary finder scrolling across a screen, e.g., “Your before meal glucose level was in range”, or the like. The screen illustrated at FIG. 6k indicates that the result was marked as after meal.

FIGS. 6l-6p illustrate a sequence of screens in a GUI in accordance with certain embodiments with marking before and after meal and summary finder on. FIG. 6m indicates selection of a before meal marker 96. FIG. 6n indicates that a reminder 98 will be provided at an interval selected by the user (e.g., 15 or 30 minutes or 1, 2 or 3 hours, or the like) 100. Then, a summary message is shown at FIG. 6o indicating that “your before meal level was 113”, or otherwise. The after-meal retest is expected after the meal. The indication of “retest” 102 may be instead “test”. FIG. 6p illustrates a countdown like that of FIG. 5i of 15 minutes during which time the user is expected to perform an after meal test.

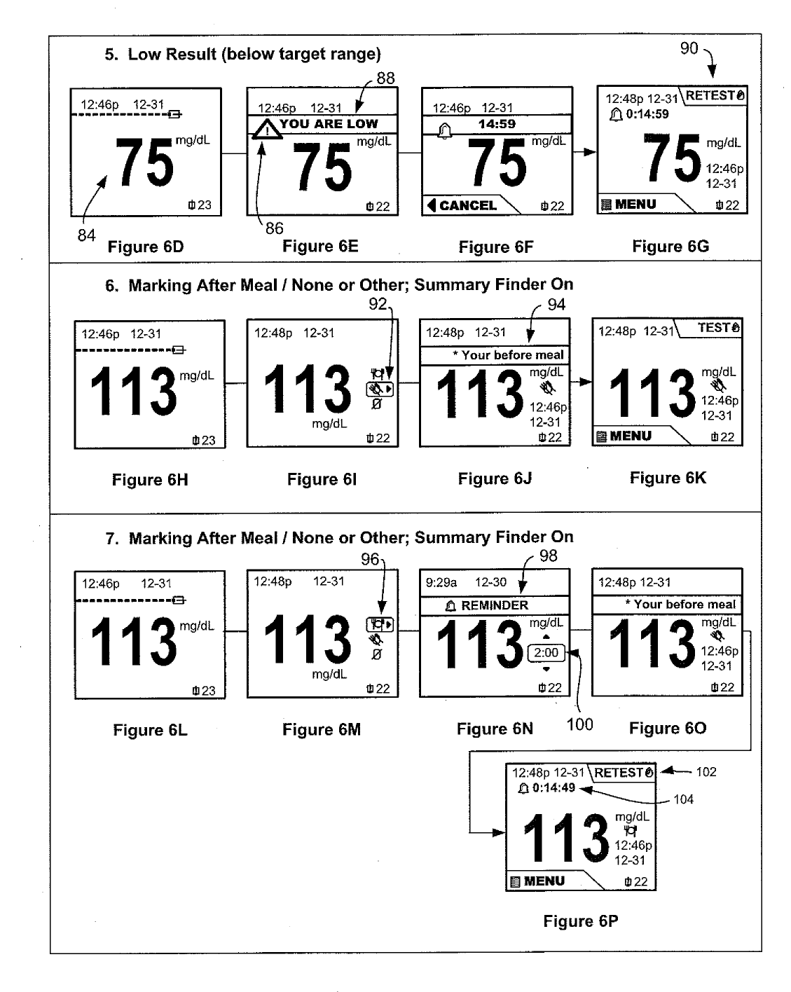

FIGS. 7a-7c indicate sequences in which a low glucose level is determined, e.g., under 20 mg/dL, which may be below the tolerance of the meter to precisely measure. FIG. 7a simply shows “Lo” for the glucose level reading as the Striplet is being recapped for ejection from the meter. FIG. 7b illustrates a screen with caution symbol flashing to warn the user that the last glucose test resulted in a seriously low reading. The screen illustrated at FIG. 7c permits the user to test or retest, particularly if the user suspects that the low reading is anomalous and not indicative of his or her actual glucose level.

FIGS. 7d-7f indicate sequences in which a high glucose level is determined, e.g., over 500 mg/dL, which may be below the tolerance of the meter to precisely measure. FIG. 7d simply shows “Hi” for the glucose level reading as the S triplet is being recapped for ejection from the meter. FIG. 7e illustrates a screen with caution symbol flashing to warn the user that the last glucose test resulted in a seriously high reading. The screen illustrated at FIG. 7f permits the user to test or retest, particularly if the user suspects that the high reading is anomalous and not indicative of his or her actual glucose level.

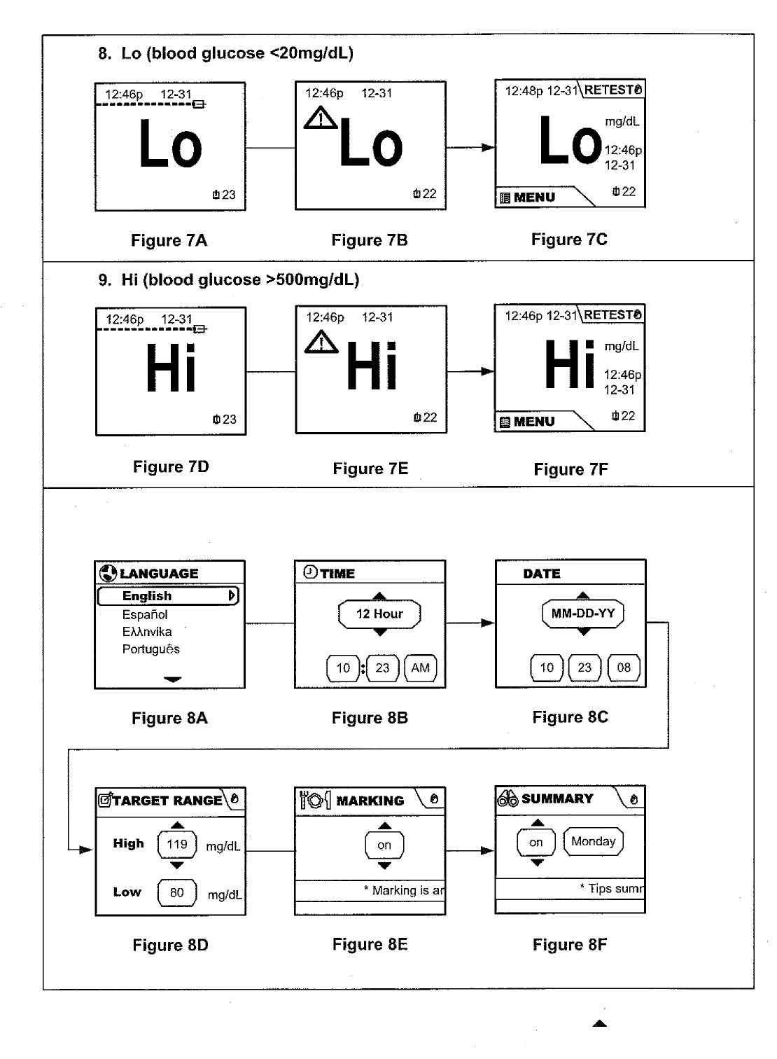

FIGS. 8a-8f illustrate screens that may appear when the user navigates through the SetUp Wizard. In FIG. 8a, a language may be selected such as English, or other language as desired. In FIG. 8b, the time may be selected as 12 hour or 24 hour time, and the particular time may be set. If the time is set and the format changed, the time will automatically convert to the corrected format. The date may be set as illustrated in FIG. 8c. Similar to time, if the date is set and the format changed, the date will automatically convert to the corrected date format. The user should keep the date and time accurate so that the analysis performed by the meter and messages provided on the screen for the user to read will be accurate, as many of these are time/date dependent. The target range can be set as illustrated at FIG. 8d. At FIG. 8e, a screen is shown wherein markers may be set to signal when a glucose test is during fasting, or before or after a meal, Scrolling text describes to the user what markers are and how to use them. At FIG. 8f, a screen is shown wherein the user is setting a summary finder for a selectable day of the week that will display a message after every blood glucose test on that day. For example, the summary may indicate on the day of the user's choosing that “you have had several low readings this week” or your readings after meals have been high this week, else they have been in range” among many other possibilities. The scrolling text in certain embodiments describes for the user what the summary is, and in a subset of those embodiments also how to use it.

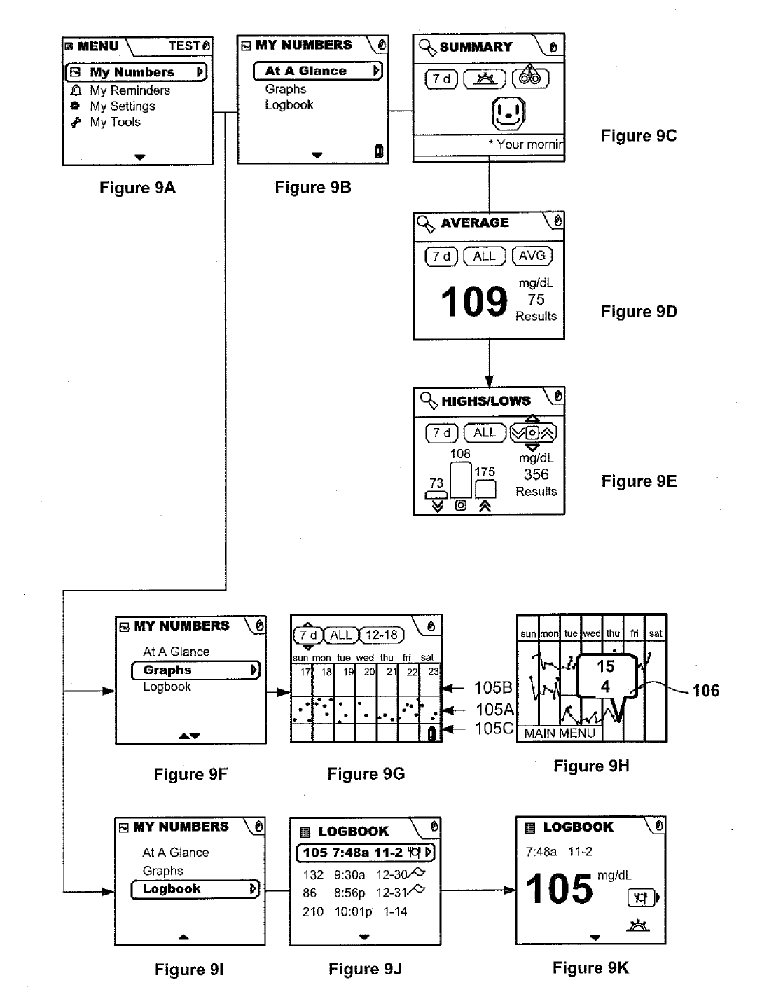

FIGS. 9a-9k illustrate screens that may appear when the user navigates through the My Numbers sub-section of a GUI for an integrated glucose meter in accordance with certain embodiments. FIG. 9a illustrates the selection of My Numbers from a home Menu screen. The screen illustrated at FIG. 9a may be pulled up whenever a user clicks the Menu icon in the lower left of the screen shown FIGS. 4h, 5b, 5e, 5i, 6c, 6g, 6p, 7c, 7f, and 9h. The user may select At a Glance, Graphs or Logbook from My Numbers as illustrated at FIG. 9b.

FIG. 9c illustrates the At a Glance screen. The At a Glance screen is configured to allow the user to view the most pertinent glucose data in one glance by choosing between various options. Along the top of the At a Glance screen are three icons in FIG. 9c. The left-most icon is a duration which can be selected at 1 day, 3 days, 7 days, 14 days, 30 or 90 days or perhaps other relevant periods. The middle icon in the top row of the At a Glance screen may indicate either all, fasting, before meal, after meal, morning, day or night may be selected, and information falling within the selected category will be presented. Further options are provided at the right-most icon in the At a Glance screen illustrated at FIG. 9c including averages, frequency counts of high, low and in range blood glucose results, and summary statements. In addition, the summary screen illustrated at FIG. 9c, which appears when At a Glance is selected from the menu of FIG. 9b, also may include an animated smiling face, caution symbol or other icon, delivering positive feedback to boost user confidence or warning of potentially dangerous blood glucose readings over time. A scrolling message may provide a most pertinent message for the user to read regarding the morning readings of the last 7 days in the example of FIG. 9c. Multiple messages may be provided. The scrolling text summarizes information for users in words in addition to numerical presentations.

FIG. 9d illustrates a screen which shows the average glucose reading for a user over a single day under all circumstances to be 109. It is also indicated that 75 readings were taken during that day.

FIG. 9e illustrates the high, low and in range readings of a user over a 7 day period under all circumstances. The screen shows that 73 results were below the selected target range, 108 were within the selected target range and 175 were above the selected target range during that week of 356 measurements.

FIG. 9f illustrates selection of graphs in the My Numbers menu. Graphs may be tailored and may be selected using the left-most icon of the top row of FIG. 9g for a certain number of days such as 1 day, 3 days, 5 days, 7 days, 10 days, 14 days, 21 days, etc. Before meals, after meals, morning, day and night or all may be selected in the middle icon of the top row of FIG. 9g. The right-most icon in the top row shows the current date. In the example of FIG. 9g, a 7 day graph is selected for measurements taken at all times during those 7 days. The graph appearing at FIG. 9g shows glucose levels versus time plotted against a calendar. The dark band 105a at the center indicates the target range, while the light band 105b above the target range indicates high glucose levels and the light band 105c below the target range indicates low glucose levels. The calendar graph delivers visual images that display fluctuations over calendar days and times.

FIG. 9h is another screen showing a 21 day graph. The user has selected a specific glucose reading on Jan. 12, 2007 at 5:57 am. The glucose reading is “154 mg/dL” for that date and time as shown in the bubble 106. The bubble 106 appearing in FIG. 9h includes certain information about the specific glucose reading that is selected. Other information might include whether the glucose was measured before or after a meal or during fasting, and there may be information regarding dosages of insulin a certain amount of time or at certain times before the reading was taken. Similar graphs can be provided for insulin dosages versus time and/or versus glucose levels.

FIG. 9i illustrates a logbook selection in the My Numbers section of the Menu. FIG. 9j illustrates a log book screen showing rows of glucose readings. Each row indicates at left the glucose reading and the time and date to the right of the glucose reading. To the right of the time and date in the log book screen of FIG. 9j is any meal marker or flag. The logbook entries can be highlighted and edited. FIG. 9k illustrates a screen that appears when a logbook entry is selected such as the top-most entry shown in FIG. 9j. FIG. 9k shows the glucose reading of “105 mg/dL”, the time and date, an icon, e.g., a rising sun, indicating that it is a morning measurement, and a before meal marker. Other indicators may include strip expired, out of temperature or invalid date/time or other dynamic feedback regarding disposable information. The GUI may be configured so that certain logbook entries are invalidated for reasons such as these and therefore not used for determining averages, graphs, or in constructing summary information tickers.

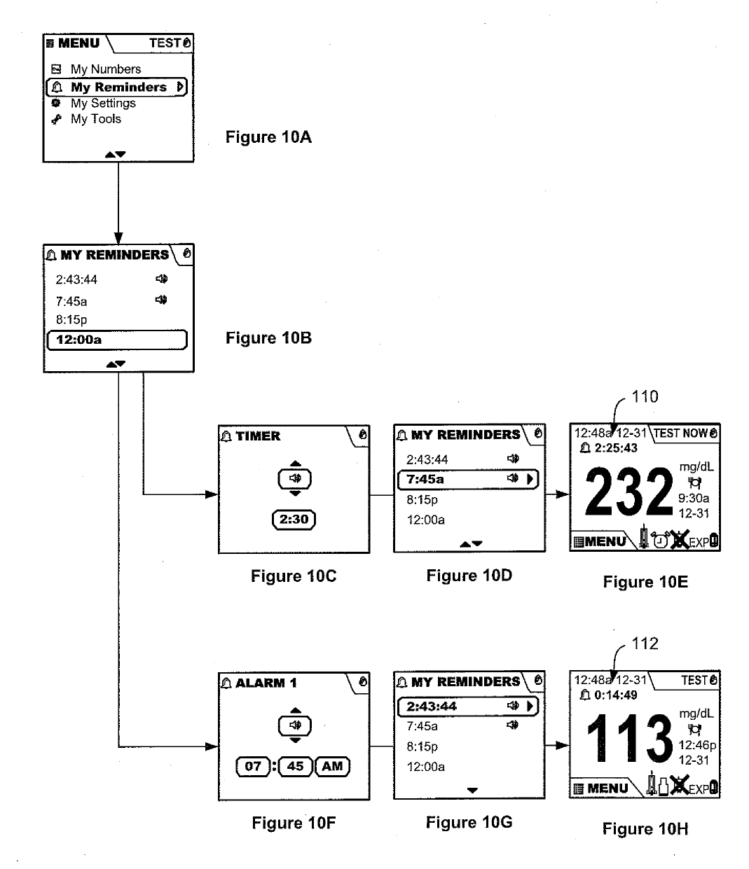

FIGS. 10a-10h illustrate navigation through the My Reminders section of a GUI for an integrated glucose meter in accordance with certain embodiments. FIG. 10a illustrates selection of My Reminders from the home screen menu. FIG. 10b illustrates several reminders that have been set by the user including a timer indicating 2:43:44 and an alarm for 7:45 am, as well as other reminders at 8:15 pm and 12:00 am. Timers and alarms help users remember to check their body glucose levels. FIG. 10c illustrates that the timing for 2:43:44 in FIG. 10b actually started at 2:30 and has run for 13 minutes and 44 second and counting. FIG. 10d shows selection of the 7:45 am reminder. FIG. 10e shows a countdown 110 to the 7:45 am alarm when the 7:45 am reminder is selected in FIG. 10d. FIG. 10f illustrates the 7:45 am alarm with volume turned down compared with the 2:30 timer which is at maximum volume. FIG. 10g illustrates selection of the 2:43:44 timer including running time indicator 112.

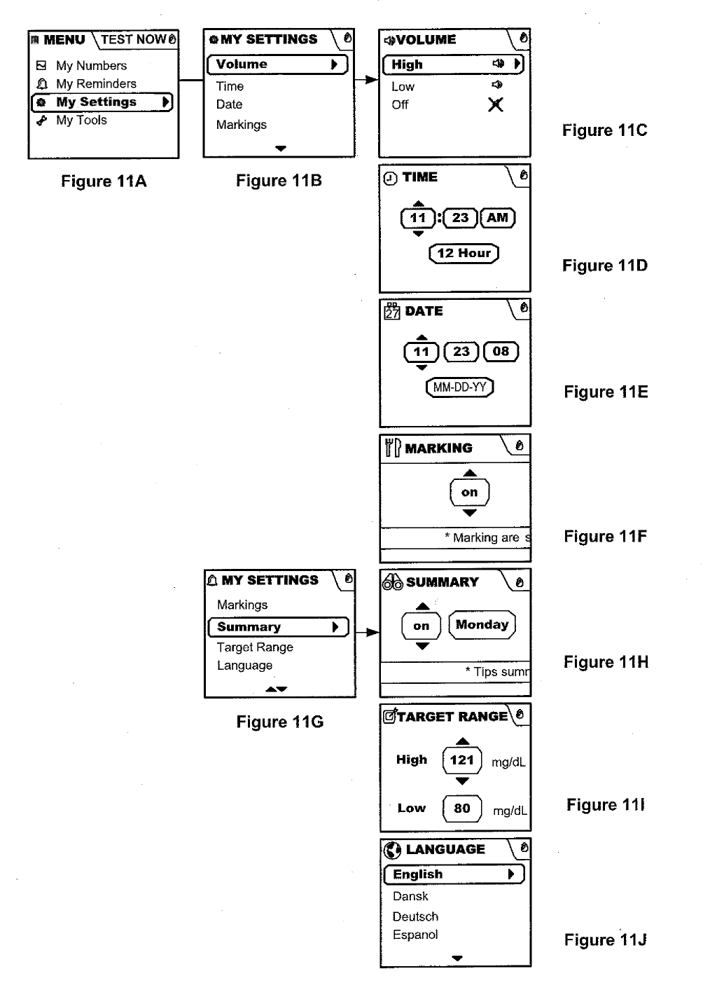

FIGS. 11a-11j illustrate screens appearing when My Settings are selected from the home screen menu of a GUI for an integrated glucose meter in accordance with certain embodiments. FIG. 11a illustrates selection of My Settings. FIGS. 11b and 11g provide various selections including volume, time, date, markings, summary, target range and language, and FIGS. 11c-11j show screens corresponding to selections of these options. The markings and summary options include scrolling tickers that describe functionality and an icon to turn off or on, while the summaries can be set for a particular day and optionally time of day, and may be set for specific information such as morning, noon or night, before or after meals, insulin dosage information or other diabetes self care information of use.

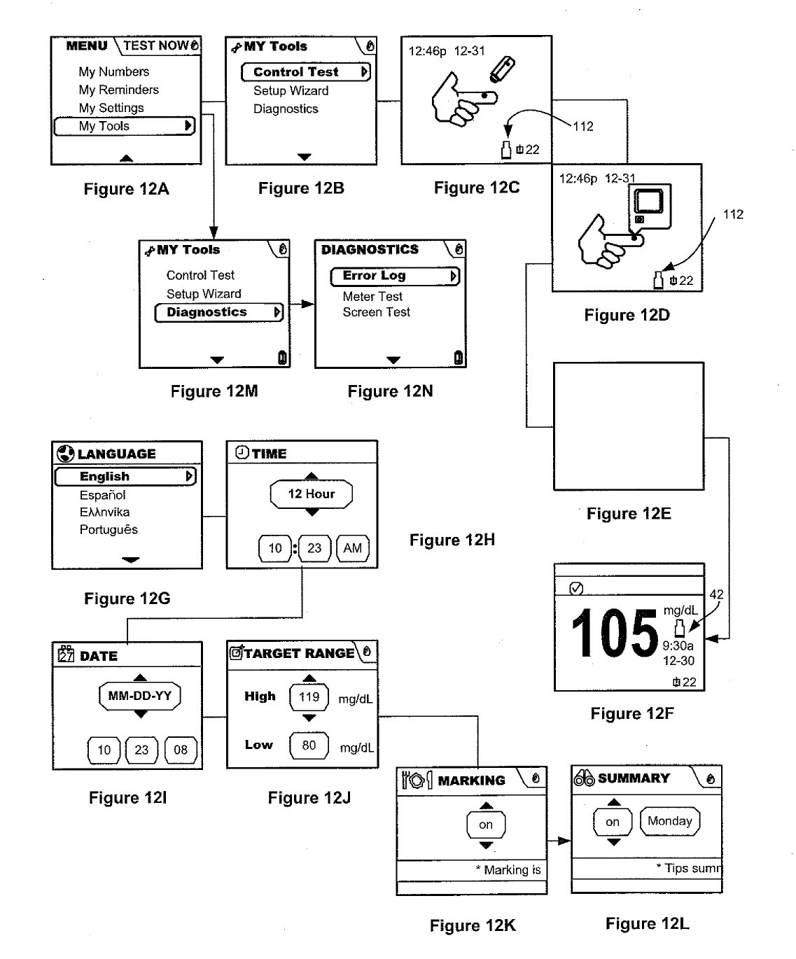

FIGS. 12a-12n illustrate screens appearing when My Tools are selected from the home screen menu of a GUI for an integrated glucose meter in accordance with certain embodiments. FIG. 12a illustrates selection of My Tools, and FIG. 12b shows that control test, setup wizard and diagnostics may be selected in My Tools.

When control test is selected, the screen of FIG. 12c may appear illustrating to a user the next step in performing a control test, which is to apply control solution on the user's finger or alternate site where a user would be lanced in a glucose test. The GUI of the meter then provides the screen of FIG. 12d illustrating the next step in the control test which is for the user to place his or her finger or alternative site at the skin receiving surface of the meter. FIG. 12e illustrates a screen indicating to the user that the meter is now performing the rest of the control test and that the user is only to continue to remain in contact with the skin receiving surface while the meter completes the test.

Advantageously, an integrated meter in accordance with certain embodiments loads a test strip in to bring the test strip into contact with the control solution on the user's finger.

The meter is programmed not to lance when a control test is being performed, while the meter would first lance before contact the testing strip to body fluid of the user. FIG. 12f illustrates that a reading is approximately what was expected from the test indicating that the meter is working properly. Throughout the control test, a bottle 112 appears on screen illustrated at FIGS. 12c, 12d and 12f indicating that a control test rather than a body glucose test is underway.

FIGS. 12g-12l illustrate screens appearing after selection of SetUp Wizard in the My Tools menu of FIG. 12b. FIG. 12g is a screen from which the language, e.g., English, is selected. FIGS. 12h and 12i are screen from which time and date are set, respectively. FIG. 12j illustrates a screen from which a target range of glucose levels is set by the user. In an alternative embodiment, the GUI may be configured such that only a doctor, e.g., with username and password, may configure the target range. FIGS. 12k-121 illustrate screens for setting markings and summary messages, each of which have been previously described. FIG. 12m illustrates a screen from which diagnostics may be selected. FIG. 12n illustrates that an error log may be accessed, particularly by a person with sophisticated knowledge of the device and what certain errors mean, and from which meter tests and screen tests may be selected and run as diagnostic tests.

While exemplary drawings and specific embodiments of the present invention have been described and illustrated, it is to be understood that that the scope of the present invention is not to be limited to the particular embodiments discussed. Thus, the embodiments shall be regarded as illustrative rather than restrictive, and it should be understood that variations may be made in those embodiments by workers skilled in the arts without departing from the scope of the present invention as set forth in the appended claims, and structural and functional equivalents thereof.

1.-69. (canceled)

70. A portable analyte monitoring apparatus, comprising:
a processor;
an electronic display in communication with the processor;
one or more user input features, and
digital memory comprising processor-readable code for programming the processor to generate and display a temporary user-readable output based on stored analyte data,
wherein the temporary user-readable output comprises multiple icons configured for manipulation by a user using the one or more user input features, including navigation between the icons and selection among multiple configurations of the icons to produce a selected display output which summarizes all or certain analyte data over a particular time period, and
wherein the selected display output comprises a scrolling word summary on a portion of the electronic display that is smaller than the complete scrolling word summary.

71. The portable analyte monitoring apparatus of claim 70, wherein the analyte is glucose.

72. The portable analyte monitoring apparatus of claim 70, wherein the electronic display is a liquid crystal display (LCD).

73. The portable analyte monitoring apparatus of claim 70, wherein the one or more user input features comprise one or more input buttons.

74. The portable analyte monitoring apparatus of claim 73, wherein the one or more input buttons are one or more touch screen buttons.

75. The portable analyte monitoring apparatus of claim 70, wherein the one or more user input features comprise a microphone.

76. The portable analyte monitoring apparatus of claim 70, further comprising a test strip receptacle configured to provide to the processor one or more signals indicative of an analyte concentration in a sample received at the test strip receptacle.

77. The portable analyte monitoring apparatus of claim 70, wherein the processor is configured to generate and display one or more feedback messages, icons or both, in connection with one or more analyte measurements.

78. The portable analyte monitoring apparatus of claim 70, wherein the analyte is glucose and the processor is configured to generate and display a notification of an analyte measurement above or below a selected target range.

79. The portable analyte monitoring apparatus of claim 70, wherein the analyte is glucose and the processor is configured to generate and display a notification of a hypoglycemic event and a reminder to test again after a predetermined time period.

80. The portable analyte monitoring apparatus of claim 70, wherein the processor is configured to generate a reminder to conduct an activity following a user-selected period of time.

81. The portable analyte monitoring apparatus of claim 80, wherein the reminder is an alarm.

82. The portable analyte monitoring apparatus of claim 80, wherein the activity is analyte testing, a meal, exercise, or insulin dosing.

83. A portable analyte monitoring apparatus, comprising:
a processor;
an electronic display in communication with the processor;
one or more user input features, and
digital memory comprising processor-readable code for programming the processor to generate and display a temporary user-readable output based on stored glucose data, and
wherein the temporary user-readable output comprises multiple icons configured for manipulation by a user, including navigation between the icons and selection among multiple configurations of the icons to produce a selected display output which summarizes all or certain analyte data over a particular time period, and
wherein a target range of analyte levels for the user is configurable by the user, and
wherein the selected display output comprises a graph showing multiple analyte measurements plotted over the time period and delineating any analyte measurements inside and outside of the target range.

84. The portable analyte monitoring apparatus of claim 83, wherein the analyte is glucose.

85. The portable analyte monitoring apparatus of claim 83, wherein the electronic display is a liquid crystal display (LCD).

86. The portable analyte monitoring apparatus of claim 83, wherein the one or more user input features comprise one or more input buttons.

87. The portable analyte monitoring apparatus of claim 86, wherein the one or more input buttons are one or more touch screen buttons.

88. The portable analyte monitoring apparatus of claim 83, wherein the one or more user input features comprise a microphone.

89. The portable analyte monitoring apparatus of claim 83, further comprising a test strip receptacle configured to provide to the processor one or more signals indicative of an analyte concentration in a sample received at the test strip receptacle.

90. The portable analyte monitoring apparatus of claim 83, wherein the processor is configured to generate and display one or more feedback messages, icons or both, in connection with one or more analyte measurements.

91. The portable analyte monitoring apparatus of claim 83, wherein the analyte is glucose and the processor is configured to generate and display a notification of an analyte measurement above or below a selected target range.

92. The portable analyte monitoring apparatus of claim 83, wherein the analyte is glucose and the processor is configured to generate and display a notification of a hypoglycemic event and a reminder to test again after a predetermined time period.

93. The portable analyte monitoring apparatus of claim 83, wherein the processor is configured to generate a reminder to conduct an activity following a user-selected period of time.

94. The portable analyte monitoring apparatus of claim 93, wherein the reminder is an alarm.

95. The portable analyte monitoring apparatus of claim 93, wherein the activity is analyte testing, a meal, exercise, or insulin dosing.

96. A portable analyte monitoring apparatus, comprising:
a processor;
an electronic display in communication with the processor;
one or more user input features, and
digital memory comprising processor-readable code for programming the processor to generate and display a temporary user-readable output based on stored analyte data, and
wherein the temporary user-readable output comprises multiple icons configured for manipulation by a user, including navigation between the icons and selection among multiple configurations of the icons to produce a selected display output which summarizes all or certain analyte data over a particular time period, and
wherein the analyte data is configurable by the user to selectably add metadata relating to conditions of particular analyte measurements, such that the selected display output is configurable by the user to summarize only certain analyte data corresponding to selected conditions.

97. The portable analyte monitoring apparatus of claim 96, wherein the electronic display is a liquid crystal display (LCD).

98. The portable analyte monitoring apparatus of claim 96, wherein the analyte is glucose.

99. The portable analyte monitoring apparatus of claim 98, wherein the metadata comprises one or more temporal relationships to one or more meals, insulin boluses, or exercise events, or time period of a day, or combinations thereof.

100. The portable analyte monitoring apparatus of claim 96, wherein the one or more user input features comprise one or more input buttons.

101. The portable diabetes care apparatus of claim 96, wherein the one or more input buttons are one or more touch screen buttons.

102. The portable diabetes care apparatus of claim 96, wherein the one or more user input features comprise a microphone.

103. The portable analyte monitoring apparatus of claim 96, further comprising a test strip receptacle configured to provide to the processor one or more signals indicative of an analyte concentration in a sample received at the test strip receptacle.

104. The portable analyte monitoring apparatus of claim 96, wherein the processor is configured to generate and display one or more feedback messages, icons or both, in connection with one or more analyte measurements.

105. The portable analyte monitoring apparatus of claim 96, wherein the analyte is glucose and the processor is configured to generate and display a notification of an analyte measurement above or below a selected target range.

106. The portable analyte monitoring apparatus of claim 96, wherein the analyte is glucose and the processor is configured to generate and display a notification of a hypoglycemic event and a reminder to test again after a predetermined time period.

107. The portable analyte monitoring apparatus of claim 96, wherein the processor is configured to generate a reminder to conduct an activity following a user-selected period of time.

108. The portable analyte monitoring apparatus of claim 107, wherein the reminder is an alarm.

109. The portable analyte monitoring apparatus of claim 107, wherein the activity is analyte testing, a meal, exercise, or insulin dosing.
